(19) United States
(12) Patent Application Publication
(10) Pub. No.: US 2026/0128142 A1
(43) Pub. Date: May 7, 2026

(54) SYSTEM AND METHOD FOR ENGAGEMENT OPERATING SYSTEM AND INDEX

(71) Applicant: Healthy Engage LLC, Frisco, TX (US)

(72) Inventor: Ravi Seshadri, Frisco, TX (US)

(21) Appl. No.: 18/939,744

(22) Filed: Nov. 7, 2024

(51) Int. Cl. G16H 20/00; G06F 16/23; G16H 10/60; G16H 40/67

(52) U.S. Cl. CPC G16H 20/00; G06F 16/2358; G16H 10/60; G16H 40/67

(57) ABSTRACT

An engagement operating system includes at least one processing device. The at least one processing device is configured to extract a plurality of data from a plurality of data sources and pre-process the plurality of data. The at least one processing device is also configured to determine an engagement index. The at least one processing device is also configured to identify, using one or more artificial intelligence models, patterns and trends using the engagement index. The at least one processing device is also configured to generate, using the identified patterns and trends, at least one of one or more engagement pathways, or one or more user experience recommendations. The at least one processing device is also configured to continuously monitor the engagement index using a monitoring and updating model to adjust at least one of the one or more engagement pathways or the one or more user experience recommendations.

### TECHNICAL FIELD

This disclosure relates generally to blockchain and machine learning systems. More specifically, this disclosure relates to a system and method for an engagement operating system and index.

### BACKGROUND

The healthcare industry is rapidly evolving and so are the options available to support it. However, current technological systems for monitoring patients and aspects of their healthcare are antiquated and rely on incomplete data and poor decision-making solutions.

### SUMMARY

This disclosure relates to a system and method for an engagement operating system and index.

In one example, an engagement operating system includes at least one processing device. The at least one processing device is configured to extract a plurality of data from a plurality of data sources and pre-process the plurality of data via transforming, filtering, modifying, and/or standardizing the plurality of data. The at least one processing device is also configured to determine an engagement index. The at least one processing device is also configured to identify, using one or more artificial intelligence models, patterns and trends using the engagement index. The at least one processing device is also configured to generate, using the identified patterns and trends, at least one of one or more engagement pathways, or one or more user experience recommendations. The at least one processing device is also configured to continuously monitor the engagement index using a monitoring and updating model to adjust at least one of the one or more engagement pathways or the one or more user experience recommendations.

In one or more of the above examples, the at least one processing device is further configured to generate an N-gram dataset using the pre-processed plurality of data and store the N-gram dataset in an N-gram repository.

In one or more of the above examples,, to generate the N-gram dataset, the at least one processing device is further configured to slide a fixed-size window over the pre-processed plurality of data to capture data combinations within the fixed-size window and create sequences of features representing a fixed-length combination of consecutive elements in the pre-processed plurality of data.

In one or more of the above examples, the at least one processing device is further configured to determine the engagement index using N-grams in the N-gram repository and identify, using the one or more artificial intelligence models, patterns and trends in the N-gram dataset.

In one or more of the above examples, the engagement index representing a numerical value reflecting a composite effect of various factors on fitness and engagement related to a user.

In one or more of the above examples, the at least one processing device is further configured to transmit data on the one or more engagement pathways to an electronic device.

In one or more of the above examples, the at least one processing device is further configured to store on a blockchain network at least a portion of one or more of the pre-processed plurality of data, the N-gram dataset, the engagement index, the one or more engagement pathways, or the one or more user experience recommendations.

In one or more of the above examples, the at least one processing device is further configured to generate an engagement dataset using the pre-processed plurality of data and create the engagement index using the engagement dataset.

In one or more of the above examples, the at least one processing device is further configured to apply the one or more user experience recommendations in one or more contexts of a plurality of contexts.

In one or more of the above examples, the at least one processing device is further configured to generate, using the identified patterns and trends, both the one or more engagement pathways and the one or more user experience recommendations.

In another example, a method of an engagement operating system includes extracting a plurality of data from a plurality of data sources and pre-processing the plurality of data via transforming, filtering, modifying, and/or standardizing the plurality of data. The method also includes determining an engagement index. The method also includes identifying, using one or more artificial intelligence models, patterns and trends using the engagement index. The method also includes generating, using the identified patterns and trends, at least one of one or more engagement pathways or one or more user experience recommendations. The method also includes continuously monitoring the engagement index using a monitoring and updating model to adjust at least one of the one or more engagement pathways or the one or more user experience recommendations.

In one or more of the above examples, the method further includes generating an N-gram dataset using the pre-processed plurality of data and storing the N-gram dataset in an N-gram repository.

In one or more of the above examples, generating the N-gram dataset includes sliding a fixed-size window over the pre-processed plurality of data to capture data combinations within the fixed-size window and creating sequences of features representing a fixed-length combination of consecutive elements in the pre-processed plurality of data.

In one or more of the above examples, the method also includes determining the engagement index using N-grams in the N-gram repository and identifying, using the one or more artificial intelligence models, patterns and trends in the N-gram dataset.

In one or more of the above examples, the engagement index representing a numerical value reflecting a composite effect of various factors on fitness and engagement related to a user.

In one or more of the above examples, the method further includes transmitting data on the one or more engagement pathways to an electronic device.

In one or more of the above examples, the method further includes storing on a blockchain network at least a portion of one or more of the pre-processed plurality of data, the N-gram dataset, the engagement index, the one or more engagement pathways, or the one or more user experience recommendations.

In one or more of the above examples, the method further includes generating an engagement dataset using the pre-processed plurality of data and creating the engagement index using the engagement dataset.

In one or more of the above examples, the method further includes applying the one or more user experience recommendations in one or more contexts of a plurality of contexts.

In one or more of the above examples, the method further includes generating, using the identified patterns and trends, both the one or more engagement pathways and the one or more user experience recommendations.

Other technical features may be readily apparent to one skilled in the art from the following figures, descriptions, and claims.

Before undertaking the DETAILED DESCRIPTION below, it may be advantageous to set forth definitions of certain words and phrases used throughout this patent document. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

As used here, terms and phrases such as “have,” “may have,” “include,” or “may include” a feature (like a number, function, operation, or component such as a part) indicate the existence of the feature and do not exclude the existence of other features. Also, as used here, the phrases “A or B,” “at least one of A and/or B,” or “one or more of A and/or B” may include all possible combinations of A and B. For example, “A or B,” “at least one of A and B,” and “at least one of A or B” may indicate all of (1) including at least one A, (2) including at least one B, or (3) including at least one A and at least one B. Further, as used here, the terms “first” and “second” may modify various components regardless of importance and do not limit the components. These terms are only used to distinguish one component from another. For example, a first user device and a second user device may indicate different user devices from each other, regardless of the order or importance of the devices. A first component may be denoted a second component and vice versa without departing from the scope of this disclosure.

It will be understood that, when an element (such as a first element) is referred to as being (operatively or communicatively) “coupled with/to” or “connected with/to” another element (such as a second element), it can be coupled or connected with/to the other element directly or via a third element. In contrast, it will be understood that, when an element (such as a first element) is referred to as being “directly coupled with/to” or “directly connected with/to” another element (such as a second element), no other element (such as a third element) intervenes between the element and the other element.

As used here, the phrase “configured (or set) to” may be interchangeably used with the phrases “suitable for,” “having the capacity to,” “designed to,” “adapted to,” “made to,” or “capable of” depending on the circumstances. The phrase “configured (or set) to” does not essentially mean “specifically designed in hardware to. ” Rather, the phrase “configured to” may mean that a device can perform an operation together with another device or parts. For example, the phrase “processor configured (or set) to perform A, B, and C” may mean a generic-purpose processor (such as a CPU or application processor) that may perform the operations by executing one or more software programs stored in a memory device or a dedicated processor (such as an embedded processor) for performing the operations.

The terms and phrases as used here are provided merely to describe some embodiments of this disclosure but not to limit the scope of other embodiments of this disclosure. It is to be understood that the singular forms “a,” “an,” and “the” include plural references unless the context clearly dictates otherwise. All terms and phrases, including technical and scientific terms and phrases, used here have the same meanings as commonly understood by one of ordinary skill in the art to which the embodiments of this disclosure belong. It will be further understood that terms and phrases, such as those defined in commonly-used dictionaries, should be interpreted as having a meaning that is consistent with their meaning in the context of the relevant art and will not be interpreted in an idealized or overly formal sense unless expressly so defined here. In some cases, the terms and phrases defined here may be interpreted to exclude embodiments of this disclosure.

Examples of an “electronic device” according to embodiments of this disclosure may include at least one of a smartphone, a tablet personal computer (PC), a mobile phone, a video phone, an e-book reader, a desktop PC, a laptop computer, a netbook computer, a workstation, a personal digital assistant (PDA), a portable multimedia player (PMP), an MP3 player, a mobile medical device, a camera, or a wearable device (such as smart glasses, a head-mounted device (HMD), electronic clothes, an electronic bracelet, an electronic necklace, an electronic accessory, an electronic tattoo, a smart mirror, or a smart watch).

Other examples of an electronic device include a smart home appliance. Examples of the smart home appliance may include at least one of a television, a digital video disc (DVD) player, an audio player, a refrigerator, an air conditioner, a cleaner, an oven, a microwave oven, a washer, a dryer, an air cleaner, a set-top box, a home automation control panel, a security control panel, a TV box (such as APPLETV or GOOGLE TV), a smart speaker or speaker with an integrated digital assistant (such as APPLE HOMEPOD or AMAZON ECHO), a gaming console (such as an XBOX, PLAYSTATION, or NINTENDO consoles), an electronic dictionary, an electronic key, a camcorder, or an electronic picture frame. Still other examples of an electronic device include at least one of various medical devices (such as diverse portable medical measuring devices (like a blood sugar measuring device, a heartbeat measuring device, or a body temperature measuring device), a magnetic resource angiography (MRA) device, a magnetic resource imaging (MRI) device, a computed tomography (CT) device, an imaging device, or an ultrasonic device), a navigation device, a global positioning system (GPS) receiver, an event data recorder (EDR), a flight data recorder (FDR), an automotive infotainment device, a sailing electronic device (such as a sailing navigation device or a gyro compass), avionics, security devices, vehicular head units, industrial or home robots, automatic teller machines (ATMs), point of sales (POS) devices, or Internet of Things (IoT) devices (such as a bulb, various sensors, electric or gas meter, sprinkler, fire alarm, thermostat, street light, toaster, fitness equipment, hot water tank, heater, or boiler). Other examples of an electronic device include at least one part of a piece of furniture or building/structure, an electronic board, an electronic signature receiving device, a projector, or various measurement devices (such as devices for measuring water, electricity, gas, or electromagnetic waves). Note that, according to various embodiments of this disclosure, an electronic device may be one or a combination of the above-listed devices. The electronic device disclosed here is not limited to the above-listed devices and may include any other electronic devices now known or later developed.

In the following description, electronic devices are described with reference to the accompanying drawings, according to various embodiments of this disclosure. As used here, the term “user” may denote a human or another device (such as an artificial intelligent electronic device) using the electronic device.

Definitions for other certain words and phrases may be provided throughout this patent document. Those of ordinary skill in the art should understand that in many if not most instances, such definitions apply to prior as well as future uses of such defined words and phrases.

None of the description in this application should be read as implying that any particular element, step, or function is an essential element that must be included in the claim scope. The scope of patented subject matter is defined only by the claims. Moreover, none of the claims is intended to invoke 35 U.S.C. § 112(f) unless the exact words “means for” are followed by a participle. Use of any other term, including without limitation “mechanism,” “module,” “device,” “unit,” “component,” “element,” “member,” “apparatus,” “machine,” “system,” “processor,” or “controller,” within a claim is understood by the Applicant to refer to structures known to those skilled in the relevant art and is not intended to invoke 35 U.S.C. § 112(f).

### BRIEF DESCRIPTION OF THE DRAWINGS

For a more complete understanding of this disclosure and its advantages, reference is now made to the following description taken in conjunction with the accompanying drawings, in which like reference numerals represent like parts:

FIG. 1 illustrates an example network configuration including an electronic device in accordance with this disclosure;

FIG. 2 illustrates an example engagement operating system architecture in accordance with this disclosure;

FIG. 3 illustrates an example method for an engagement operating system in accordance with this disclosure;

FIG. 4 illustrates an example engagement index system architecture in accordance with this disclosure; and

FIG. 5 illustrates an example engagement index method in accordance with this disclosure.

### DETAILED DESCRIPTION

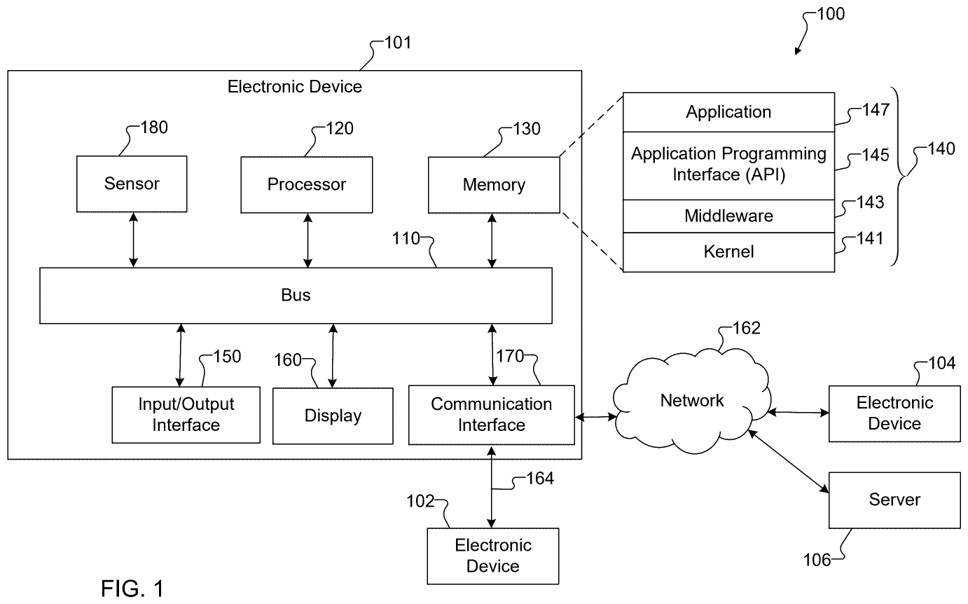

FIGS. 1 through 5, discussed below, and the various embodiments of this disclosure are described with reference to the accompanying drawings. However, it should be appreciated that this disclosure is not limited to these embodiments, and all changes and/or equivalents or replacements thereto also belong to the scope of this disclosure. The same or similar reference denotations may be used to refer to the same or similar elements throughout the specification and the drawings.

As noted above, the healthcare industry is rapidly evolving and so are the options available to support it. However, current technological systems for monitoring patients and aspects of their healthcare are antiquated and rely on incomplete data and poor decision-making solutions.

In various embodiments, this disclosure provides an engagement operating system (EOS) and methods for facilitating the EOS. As one example, this disclosure provides for extracting data into an executable engines that includes a virtual environment, analyzing engagement pathways, generating an N-gram dataset (a collection of n successive items) comprising data indicative and predictive of fitness of an individual's journey, compressed in an N-gram from a comprehensive dataset according to a data structure indicative and predictive of fitness of the individual, the data structure including a numerical index representing a composite effect of various environmental, social, and contextual conditions of the individual including interdependencies of the health conditions, generating an N-gram based on the N-gram dataset, and calculating the individual's fitness using the N-gram.

The EOS and associated methods utilize artificial intelligence, a blockchain system, and engagement index measurements to analyze and enhance individual engagement in various aspects such as health, personal development, and social interactions. The EOS extracts data from multiple sources, creates an N-gram dataset, and generates an engagement index based on social, environmental, attitudinal, and behavioral data segments. The EOS also utilizes advanced algorithms and data structures to predict and optimize individual fitness and engagement levels. n various embodiments, the EOS extracts data from various sources, including social media, environmental sensors, personal devices, and user-generated content, to create the N-gram dataset and calculate the individual's engagement index. As noted above, the engagement index represents a composite effect of various factors, such as environmental, social interactions, attitudes, and behaviors.

In various embodiments, this disclosure also provides systems and methods for identifying engagement of a consumer in a particular context (e.g., healthcare provider, payer, etc.) over time by integrating the consumer's behavior in different online mediums and physical interactions (such as captured through surveys) with the use of algorithms that measure affective (a person's way of feeling or expressing emotions), behavioral, and/or cognitive attributes (such as knowledge of the engagement activity) and captured with attributions such as intensity, frequency, recency, and duration.

FIG. 1 illustrates an example network configuration 100 including an electronic device in accordance with this disclosure. The embodiment of the network configuration 100 shown in FIG. 1 is for illustration only. Other embodiments of the network configuration 100 could be used without departing from the scope of this disclosure.

According to embodiments of this disclosure, an electronic device 101 is included in the network configuration 100. The electronic device 101 can include at least one of a bus 110, a processor 120, a memory 130, an input/output (I/O) interface 150, a display 160, a communication interface 170, or a sensor 180. In some embodiments, the electronic device 101 may exclude at least one of these components or may add at least one other component. The bus 110 includes a circuit for connecting the components 120-180 with one another and for transferring communications (such as control messages and/or data) between the components.

The processor 120 includes one or more processing devices, such as one or more microprocessors, microcontrollers, digital signal processors (DSPs), application specific integrated circuits (ASICs), or field programmable gate arrays (FPGAs). In some embodiments, the processor 120 includes one or more of a central processing unit (CPU), an application processor (AP), a communication processor (CP), or a graphics processor unit (GPU). The processor 120 is able to perform control on at least one of the other components of the electronic device 101 and/or perform an operation or data processing relating to communication or other functions.

The memory 130 can include a volatile and/or non-volatile memory. For example, the memory 130 can store commands or data related to at least one other component of the electronic device 101. According to embodiments of this disclosure, the memory 130 can store software and/or a program 140. The program 140 includes, for example, a kernel 141, middleware 143, an application programming interface (API) 145, and/or an application program (or “application”) 147. At least a portion of the kernel 141, middleware 143, or API 145 may be denoted an operating system (OS).

The kernel 141 can control or manage system resources (such as the bus 110, processor 120, or memory 130) used to perform operations or functions implemented in other programs (such as the middleware 143, API 145, or application 147). The kernel 141 provides an interface that allows the middleware 143, the API 145, or the application 147 to access the individual components of the electronic device 101 to control or manage the system resources. These functions can be performed by a single application or by multiple applications that each carries out one or more of these functions. The middleware 143 can function as a relay to allow the API 145 or the application 147 to communicate data with the kernel 141, for instance. A plurality of applications 147 can be provided. The middleware 143 is able to control work requests received from the applications 147, such as by allocating the priority of using the system resources of the electronic device 101 (like the bus 110, the processor 120, or the memory 130) to at least one of the plurality of applications 147. The API 145 is an interface allowing the application 147 to control functions provided from the kernel 141 or the middleware 143.

The I/O interface 150 serves as an interface that can, for example, transfer commands or data input from a user or other external devices to other component(s) of the electronic device 101. The I/O interface 150 can also output commands or data received from other component(s) of the electronic device 101 to the user or the other external device.

The display 160 includes, for example, a liquid crystal display (LCD), a light emitting diode (LED) display, an organic light emitting diode (OLED) display, a quantum-dot light emitting diode (QLED) display, a microelectromechanical systems (MEMS) display, or an electronic paper display. The display 160 can also be a depth-aware display, such as a multi-focal display. The display 160 is able to display, for example, various contents (such as text, images, videos, icons, or symbols) to the user. The display 160 can include a touchscreen and may receive, for example, a touch, gesture, proximity, or hovering input using an electronic pen or a body portion of the user.

The communication interface 170, for example, is able to set up communication between the electronic device 101 and an external electronic device (such as a first electronic device 102, a second electronic device 104, or a server 106). For example, the communication interface 170 can be connected with a network 162 or 164 through wireless or wired communication to communicate with the external electronic device. The communication interface 170 can be a wired or wireless transceiver or any other component for transmitting and receiving signals, such as images.

The electronic device 101 further includes one or more sensors 180 that can meter a physical quantity or detect an activation state of the electronic device 101 and convert metered or detected information into an electrical signal. For example, one or more sensors 180 can include one or more cameras or other imaging sensors for capturing images of scenes. The sensor(s) 180 can also include one or more buttons for touch input, one or more microphones, a gesture sensor, a gyroscope or gyro sensor, an air pressure sensor, a magnetic sensor or magnetometer, an acceleration sensor or accelerometer, a grip sensor, a proximity sensor, a color sensor (such as an RGB sensor), a bio-physical sensor, a temperature sensor, a humidity sensor, an illumination sensor, an ultraviolet (UV) sensor, an electromyography (EMG) sensor, an electroencephalogram (EEG) sensor, an electrocardiogram (ECG) sensor, an infrared (IR) sensor, an ultrasound sensor, an iris sensor, or a fingerprint sensor. The sensor(s) 180 can further include an inertial measurement unit, which can include one or more accelerometers, gyroscopes, and other components. In addition, the sensor(s) 180 can include a control circuit for controlling at least one of the sensors included here. Any of these sensor(s) 180 can be located within the electronic device 101.

The first external electronic device 102 or the second external electronic device 104 can be a wearable device or an electronic device-mountable wearable device (such as an HMD). When the electronic device 101 is mounted in the electronic device 102 (such as the HMD), the electronic device 101 can communicate with the electronic device 102 through the communication interface 170. The electronic device 101 can be directly connected with the electronic device 102 to communicate with the electronic device 102 without involving with a separate network. The electronic device 101 can also be an augmented reality wearable device, such as eyeglasses, that include one or more cameras.

The wireless communication is able to use at least one of, for example, long term evolution (LTE), long term evolution-advanced (LTE-A), 5th generation wireless system (5G), millimeter-wave or 60 GHz wireless communication, Wireless USB, code division multiple access (CDMA), wideband code division multiple access (WCDMA), universal mobile telecommunication system (UMTS), wireless broadband (WiBro), or global system for mobile communication (GSM), as a cellular communication protocol. The wired connection can include, for example, at least one of a universal serial bus (USB), high definition multimedia interface (HDMI), recommended standard 232 (RS-232), or plain old telephone service (POTS). The network 162 includes at least one communication network, such as a computer network (like a local area network (LAN) or wide area network (WAN)), Internet, or a telephone network.

The first and second external electronic devices 102 and 104 and server 106 each can be a device of the same or a different type from the electronic device 101. According to certain embodiments of this disclosure, the server 106 includes a group of one or more servers. Also, according to certain embodiments of this disclosure, all or some of the operations executed on the electronic device 101 can be executed on another or multiple other electronic devices (such as the electronic devices 102 and 104 or server 106). Further, according to certain embodiments of this disclosure, when the electronic device 101 should perform some function or service automatically or at a request, the electronic device 101, instead of executing the function or service on its own or additionally, can request another device (such as electronic devices 102 and 104 or server 106) to perform at least some functions associated therewith.

The other electronic device (such as electronic devices 102 and 104 or server 106) is able to execute the requested functions or additional functions and transfer a result of the execution to the electronic device 101. The electronic device 101 can provide a requested function or service by processing the received result as it is or additionally. To that end, a cloud computing, distributed computing, or client-server computing technique may be used, for example. While FIG. 1 shows that the electronic device 101 includes the communication interface 170 to communicate with the external electronic device 104 or server 106 via the network 162, the electronic device 101 may be independently operated without a separate communication function according to some embodiments of this disclosure.

The server 106 can include the same or similar components as the electronic device 101 (or a suitable subset thereof). The server 106 can support to drive the electronic device 101 by performing at least one of operations (or functions) implemented on the electronic device 101. For example, the server 106 can include a processing module or processor that may support the processor 120 implemented in the electronic device 101.

Although FIG. 1 illustrates one example of a network configuration 100 including an electronic device 101, various changes may be made to FIG. 1. For example, the network configuration 100 could include any suitable number of each component in any suitable arrangement. In general, computing and communication systems come in a wide variety of configurations, and FIG. 1 does not limit the scope of this disclosure to any particular configuration. Also, while FIG. 1 illustrates one operational environment in which various features disclosed in this patent document can be used, these features could be used in any other suitable system.

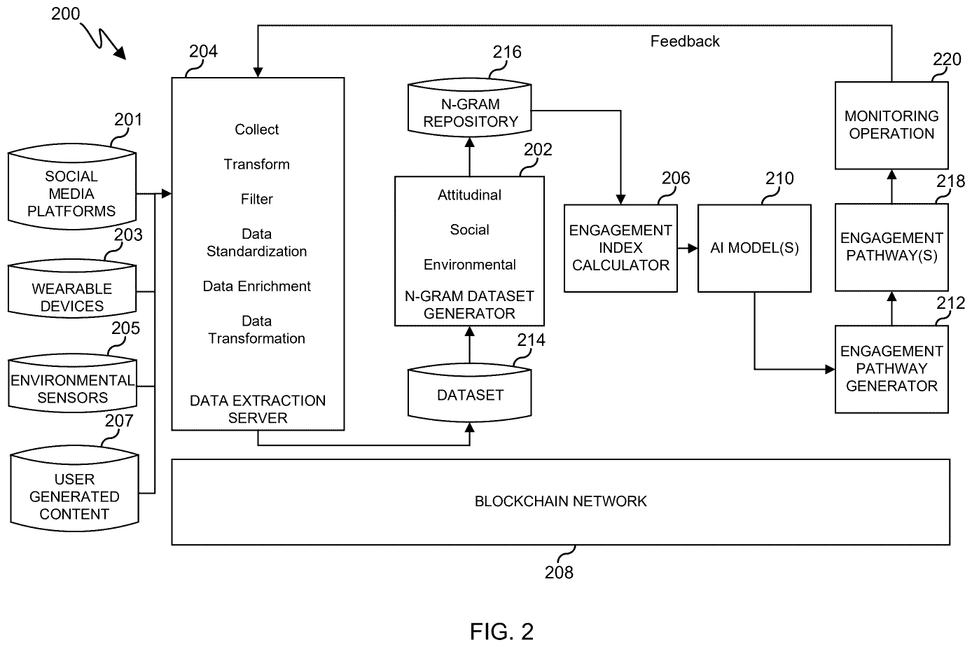

FIG. 2 illustrates an example engagement operating system architecture 200 in accordance with this disclosure. For ease of explanation, the architecture 200 shown in FIG. 2 is described as being implemented on or supported by the electronic device 101 in the network configuration 100 of FIG. 1. However, the architecture 200 shown in FIG. 2 could be used with any other suitable device(s) and in any other suitable system(s), such as when the architecture 200 is implemented on or supported by the server 106.

The engagement operating system (EOS) architecture 200 utilizes artificial intelligence and blockchain technology and engagement index measurements to analyze and enhance individual engagement in various aspects, including health, personal development, and social interactions. The EOS extracts data from multiple sources, creates an N-gram dataset, and generates an engagement index based on social, environmental, attitudinal, and behavioral data segments. The EOS also utilizes advanced algorithms and data structures to predict and optimize individual fitness and engagement levels.

For example, as shown in FIG. 2, the architecture 200 includes an N-gram dataset generator 202, a data extraction server 204, an engagement index calculator 206, a blockchain network 208, one or more artificial intelligence models 210, and an engagement pathway generator 212. The data extraction server 204 can be connected to various data sources including social media platform sources 201, wearable device sources 203, environmental sources 205 (such as IoT devices), and user generated content sources 207. The data sources may have their data stored in the form of databases. The data extraction server 204 can be a dedicated server responsible for extracting relevant data from various sources. The data extraction server 204, using the various data sources, collects, transforms and/or filters the data, and/or performs data standardization, data enrichment, and/or data transformation on the data. For example, the data extraction server 204 performs data acquisition and normalization by collecting data from the multiple sources and performs data preprocessing techniques such as data cleaning, transformation, and normalization to ensure a consistent format and representation across different sources. Using the data extraction server 204, the architecture 200 gathers data from the multiple sources and generates a comprehensive dataset 214 for each subject individual.

In various embodiments, data collection performed by the data extraction server 204 involves the data extraction server 204 connecting to various data sources, such as databases, APIs, IoT devices, and third-party services, to collect the data for the EOS. In various embodiments, data transformation performed by the data extraction server 204 involves the data extraction server 204 transforming the collected data into a unified format compatible with other components of the architecture 200, such as the N-gram dataset generator 202, the engagement index calculator 206, and the one or more artificial intelligence models 210. This can include parsing, decoding, and converting data to ensure seamless integration with the rest of the system.

In various embodiments, data filtering performed by the data extraction server 204 involves the data extraction server 204 filtering the collected data based on predefined criteria or rules, ensuring that only relevant and high-quality data is passed on for further analysis to other components of the architecture 200, such as the N-gram dataset generator 202, the engagement index calculator 206, and the one or more artificial intelligence models 210. In various embodiments, data enrichment performed by the data extraction server 204 involves the data extraction server 204 enriching the collected data with additional information from external sources, if required, to enhance the context and accuracy of the data fed into the other components of the architecture 200. In various embodiments, data transmission performed by the data extraction server 204 involves the data extraction server 204 securely transmitting the preprocessed data to the other components of the architecture 200.

The pre-processing of the data by the data extraction server 204 thus allows for, before creating the N-gram dataset, the raw data to be collected from the multiple sources and cleaned, transformed, and structured into a format suitable for analysis. The data extraction server 204 may also address missing or incomplete data, such as removing outliers and/or normalizing values to ensure consistent representation across data sources.

The processed data, such as the data in the dataset 214, can be securely stored on the blockchain network 208, ensuring data integrity, immutability, and traceability. Other data created by components of the architecture 200, such as the N-gram dataset generator 202, the engagement index calculator 206, and the one or more artificial intelligence models 210 can also be stored in the blockchain network 208. The blockchain network 208 thus allows for securely storing and sharing data, and ensuring data integrity, privacy, and compliance with consent and legal requirements. The blockchain network 208 also facilitates trust and transparency among EOS architecture 200 users and stakeholders.

The EOS architecture 200 utilizes advanced algorithms to analyze the collected data in the dataset 214 to generate an N-gram dataset using the N-gram dataset generator 202. N-grams created using the N-gram dataset generator 202 include data indicative and predictive of an individual's fitness and engagement levels. The N-gram dataset captures the complex interdependencies between various factors, including environmental, social, attitudinal, and behavioral data segments. The N-gram dataset generator 202 is configured to analyze individual engagement and fitness and create an N-gram dataset from the comprehensive dataset 214 collected from various data sources. N-grams created using the N-gram dataset generator 202 can be stored in a storage location, such as an N-gram repository 216.

For example, to create the N-gram dataset by the N-gram dataset generator 202, the N-gram dataset generator 202 performs feature extraction to identify relevant features within the comprehensive dataset 214 that contribute to individual fitness and engagement levels. These features can include social interactions, environmental factors, personal attitudes, behaviors, and other variables that impact overall well-being. This feature extraction performed by the N-gram dataset generator 202 may involve techniques such as Principal Component Analysis (PCA) or other dimensionality reduction methods to identify the most influential variables.

In various embodiments, the N-gram dataset is constructed by the N-gram dataset generator 202 by analyzing the structured data and creating sequences of features, where each sequence represents a fixed-length combination of consecutive elements (e.g., words, actions, or states). The N-grams are generated by sliding a fixed-size window over the data and capturing all possible combinations within that window. For example, if N=3 (trigram), the system would capture all possible three-element sequences within the dataset 214.

Based on the N-gram dataset created by the N-gram dataset generator 202, the EOS architecture 200, using the engagement index calculator 206, calculates an individual's engagement index based on the generated N-gram dataset. The engagement index represents a numerical value reflecting the composite effect of various factors on an individual's overall fitness and engagement. The one or more artificial intelligence models 210 are configured to perform, using the engagement index, various machine learning techniques to identify patterns and trends in the N-gram dataset, predict future engagement levels, and optimize individual fitness. This analysis helps tailor personalized engagement pathways and strategies for each user.

In various embodiments, the one or more artificial intelligence models 210 utilize various techniques to analyze the N-gram dataset and identify patterns, trends, and relationships between features. For example, the one or more artificial intelligence models 210 can include N-gram Language Models that are configured to estimate the probability of observing a specific sequence of features in the dataset, which can be used to predict future engagement levels and help identify optimal engagement pathways. The one or more artificial intelligence models 210 can also include neural networks that are configured to perform deep learning techniques, such as recurrent neural networks (RNNs) or long short-term memory (LSTM) networks, which can be applied to the N-gram dataset to capture complex dependencies and temporal patterns in the data.

As another example, the one or more artificial intelligence models 210 can perform clustering techniques, such as K-means clustering or density-based spatial clustering of applications with noise (DBSCAN) techniques, to group individuals with similar engagement patterns and behaviors, allowing for the development of tailored engagement strategies. The one or more artificial intelligence models 210 can also perform association rule learning, which can be algorithms that can be employed to discover frequent co-occurring feature combinations in the N-gram dataset, providing insights into common engagement patterns and potential intervention points. In various embodiments, the N-gram dataset can be continuously updated as new data becomes available, ensuring that the AI-driven analysis and engagement pathways remain relevant and adaptive to changing individual needs and preferences.

Outputs from the one or more artificial intelligence models 210 are provided to the engagement pathway generator 212. Based on the AI-driven analysis, the EOS architecture 200 generates, using the engagement pathway generator 212, engagement pathways 218 tailored to each individual, addressing their unique needs and preferences. The engagement pathways 218 can include personalized recommendations, goals, and interventions to enhance engagement of the individual with their healthcare, for example, and their overall well-being.

The architecture 200 also provides for a continuous monitoring operation 220 that continuously monitors user progress and adjusts the engagement pathways 218 based on real-time data and feedback. The monitoring operation 220 creates a feedback loop that enables adaptive and dynamic engagement strategies that evolve with individual needs and preferences. In some embodiments, the monitoring operation 220 can also be used to track data for use in updating or retraining the one or more artificial intelligence models 210. In various embodiments, the EOS architecture can incorporate gamification elements and incentives to motivate users and encourage sustained engagement. This can include rewards, achievements, and social recognition for reaching milestones or completing challenges.

In various embodiments, the EOS architecture 200 can be configured to evaluate engagement in the context of healthcare by considering factors such as patient adherence to treatment plans, regularity of medical appointments, participation in wellness programs, and responsiveness to healthcare provider communications. By analyzing these factors, the EOS architecture 200 assists with identifying areas for improvement and tailoring personalized healthcare engagement strategies for individuals, ultimately leading to better health outcomes and increased patient satisfaction. Additionally or alternatively, the EOS architecture 200 can also integrate additional data sources relevant to patient engagement, such as electronic health records (EHRs), patient-reported outcome measures (PROMs), data from telehealth platforms, and information from patient support groups. These additional data sources can provide a more comprehensive view of an individual's engagement in their healthcare journey, allowing for more accurate predictions and tailored interventions.

Additionally or alternatively, the engagement index calculator 206 can be expanded to incorporate healthcare-specific factors when calculating an individual's engagement index. These factors can include, for example, adherence to treatment plans, such as the degree to which a patient follows the prescribed treatment plan, including medication intake, therapy sessions, and lifestyle changes, and/or attendance at medical appointments, such as the regularity of a patient's attendance at medical appointments, both for routine check-ups and follow-up visits related to ongoing conditions, participation in wellness programs, such as the extent to which a patient engages in wellness programs or preventative healthcare initiatives, such as exercise programs, nutrition counseling, or mental health support. In various embodiments, these factors can include, additionally or alternatively, responsiveness to healthcare provider communications, such as how promptly and consistently a patient responds to communications from healthcare providers, such as appointment reminders, test result notifications, or educational materials, patient-reported outcomes, such as PROMs, that can provide insights into a patient's subjective experience of their health condition, treatment effectiveness, and overall quality of life, and/or social support and peer interactions, such as the level of support an individual receives from friends, family, or support groups, as well as their participation in peer-to-peer interactions, such as online forums or community events.

Although FIG. 2 illustrates one example of an EOS architecture 200, various changes may be made to FIG. 2. For example, various components and functions in FIG. 2 may be combined, further subdivided, replicated, or rearranged according to particular needs. Also, one or more additional components and functions may be included if needed or desired. Further, various components shown in FIG. 2, such as the N-gram dataset generator 202, the engagement index calculator 206, and the engagement pathway generator 212, can be executed by specific electronic devices configured to perform functions related to those components of the architecture 200, or can represent software operations executed by one or more electronic devices.

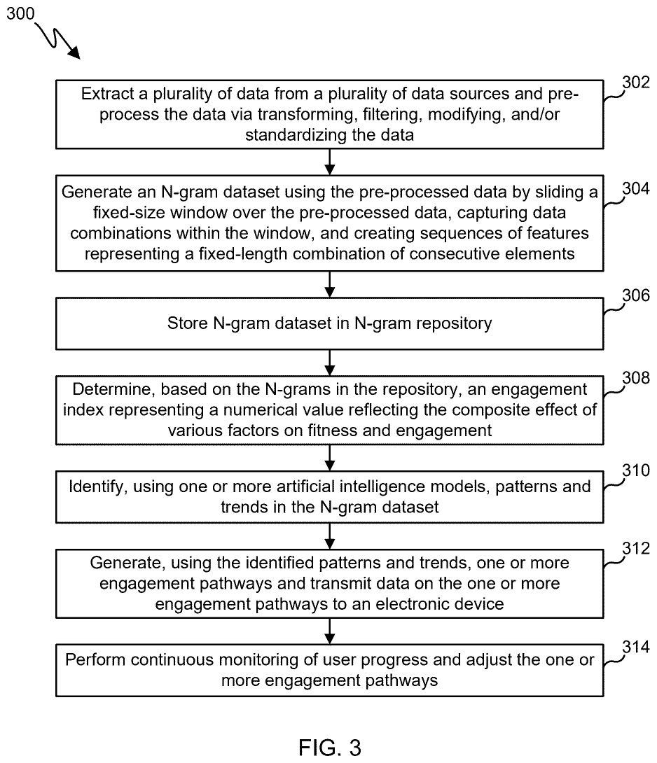

FIG. 3 illustrates an example method 300 for an engagement operating system in accordance with this disclosure. For ease of explanation, the method 300 shown in FIG. 3 is described as being performed using the electronic device 101 in the network configuration 100 of FIG. 1. However, the method 300 could be performed using any other suitable device(s), such as the server 106, and in any other suitable system(s).

At step 302, at least one processing device extracts a plurality of data from a plurality of data sources and transforms, filters, modifies, and/or standardizes the data. For example, as described with respect to FIG. 2, the data extraction server 204 can be used to extract the data from sources, such as the sources 201, 203, 205, 207 of FIG. 2, and perform the manipulation of the data. In some embodiments, the processed data can be stored in a dataset, such as the dataset 214 of FIG. 2. For example, the data pre-processing at step 302 can include techniques such as data cleaning, transformation, and normalization to ensure a consistent format and representation across different sources. In this way, data can be gathered from the multiple sources and a comprehensive dataset can be generated for each subject individual.

At step 304, the at least one processing device generates an N-gram dataset, such as by using the N-gram dataset generator 202 of FIG. 2. The N-grams of this disclosure include data indicative and predictive of an individual's fitness and engagement levels. The N-gram dataset captures the complex interdependencies between various factors, including environmental, social, attitudinal, and behavioral data segments. Generating the N-gram dataset can include analyzing individual engagement and fitness and create the N-gram dataset from a comprehensive dataset that includes the pre-processed data collected from various data sources.

For example, to create the N-gram dataset, the at least one processing device can perform feature extraction to identify relevant features within the comprehensive dataset that contribute to individual fitness and engagement levels. These features can include social interactions, environmental factors, personal attitudes, behaviors, and other variables that impact overall well-being. This feature extraction may involve techniques such as PCA or other dimensionality reduction methods to identify the most influential variables. In various embodiments, the N-gram dataset is constructed by analyzing the structured data and creating sequences of features, where each sequence represents a fixed-length combination of consecutive elements (e.g., words, actions, or states). This can include sliding a fixed-size window over the data and capturing all possible combinations within that window. For example, if N=3 (trigram), the system would capture all possible three-element sequences within the dataset. At step 306, the N-grams created at step 304 can be stored in an N-gram repository, such as the N-gram repository 216.

Based on the N-gram dataset in the repository, at step 308, the at least one processing device determines, such as by using the engagement index calculator 206, an individual's engagement index based on the generated N-gram dataset. The engagement index represents a numerical value reflecting the composite effect of various factors on an individual's overall fitness and engagement. At step 308, the at least one processing device identifies patterns and trends in the N-gram dataset using one or more artificial intelligence models, such as the one or more artificial intelligence models 210. In various embodiments, the one or more artificial ingelligence models are configured to perform, using the engagement index, various machine learning techniques to identify patterns and trends in the N-gram dataset, predict future engagement levels, and optimize individual fitness. This analysis helps tailor personalized engagement pathways and strategies for each user.

In various embodiments, the one or more artificial intelligence models utilize various techniques to analyze the N-gram dataset and identify patterns, trends, and relationships between features. For example, the one or more artificial intelligence models can include N-gram Language Models that are configured to estimate the probability of observing a specific sequence of features in the dataset, which can be used to predict future engagement levels and help identify optimal engagement pathways. The one or more artificial intelligence models can also include neural networks that are configured to perform deep learning techniques, such as RNNs or LSTM networks, which can be applied to the N-gram dataset to capture complex dependencies and temporal patterns in the data.

As another example, the one or more artificial intelligence models can perform clustering techniques, such as K-means clustering or DBSCAN techniques, to group individuals with similar engagement patterns and behaviors, allowing for the development of tailored engagement strategies. The one or more artificial intelligence models can also perform association rule learning, which can be algorithms that can be employed to discover frequent co-occurring feature combinations in the N-gram dataset, providing insights into common engagement patterns and potential intervention points. In various embodiments, the N-gram dataset can be continuously updated as new data becomes available, ensuring that the AI-driven analysis and engagement pathways remain relevant and adaptive to changing individual needs and preferences.

At step 312, the at least one processing device generates, using the identified patterns and trends determined using the one or more artificial intelligence models, one or more engagement pathways and transmit data on the one or more engagement pathways to an electronic device. For example, outputs from the one or more artificial intelligence models can be provided to an engagement pathway generator, such as the engagement pathway generator 212. Based on the AI-driven analysis, engagement pathways are generated that are tailored to each individual, addressing their unique needs and preferences. The engagement pathways can include personalized recommendations, goals, and interventions to enhance engagement of the individual with their healthcare, for example, and their overall well-being.

At step 314, the at least one processing device performs continuous monitoring of user progress and adjust the one or more engagement pathways. For example, as described with respect to FIG. 2, a continuous monitoring operation, such as the continuous monitoring operation 220, can be used to continuously monitor user progress and adjust the engagement pathways based on real-time data and feedback. The monitoring operation creates a feedback loop that enables adaptive and dynamic engagement strategies that evolve with individual needs and preferences. In some embodiments, the monitoring operation can also be used to track data for use in updating or retraining the one or more artificial intelligence models. In various embodiments, gamification elements and incentives can also be incorporated to motivate users and encourage sustained engagement. This can include rewards, achievements, and social recognition for reaching milestones or completing challenges.

In various embodiments, the method 300 can also include evaluating engagement in the context of healthcare by considering factors such as patient adherence to treatment plans, regularity of medical appointments, participation in wellness programs, and responsiveness to healthcare provider communications. By analyzing these factors, the method 300 assists with identifying areas for improvement and tailoring personalized healthcare engagement strategies for individuals, ultimately leading to better health outcomes and increased patient satisfaction. Additionally or alternatively, the method 300 can also integrate additional data sources relevant to patient engagement, such as EHRs, PROMs, data from telehealth platforms, and information from patient support groups. These additional data sources can provide a more comprehensive view of an individual's engagement in their healthcare journey, allowing for more accurate predictions and tailored interventions.

Additionally or alternatively, the calculation of the engagement index can be expanded to incorporate healthcare-specific factors when calculating an individual's engagement index. These factors can include, for example, adherence to treatment plans, such as the degree to which a patient follows the prescribed treatment plan, including medication intake, therapy sessions, and lifestyle changes, and/or attendance at medical appointments, such as the regularity of a patient's attendance at medical appointments, both for routine check-ups and follow-up visits related to ongoing conditions, participation in wellness programs, such as the extent to which a patient engages in wellness programs or preventative healthcare initiatives, such as exercise programs, nutrition counseling, or mental health support. In various embodiments, these factors can include, additionally or alternatively, responsiveness to healthcare provider communications, such as how promptly and consistently a patient responds to communications from healthcare providers, such as appointment reminders, test result notifications, or educational materials, patient-reported outcomes, such as PROMs, that can provide insights into a patient's subjective experience of their health condition, treatment effectiveness, and overall quality of life, and/or social support and peer interactions, such as the level of support an individual receives from friends, family, or support groups, as well as their participation in peer-to-peer interactions, such as online forums or community events.

In various embodiments, the method 300 can also include securely storing the processed data, such as the data in the dataset 214, using a blockchain network, such as the blockchain network 208, ensuring data integrity, immutability, and traceability. Other data created, such as the N-gram dataset, the engagement index, and outputs of the one or more artificial intelligence models, can also be stored in the blockchain network.

Although FIG. 3 illustrates one example of a method 300 for an engagement operating system, various changes may be made to FIG. 3. For example, while shown as a series of steps, various steps in FIG. 3 could overlap, occur in parallel, occur in a different order, or occur any number of times (including zero times).

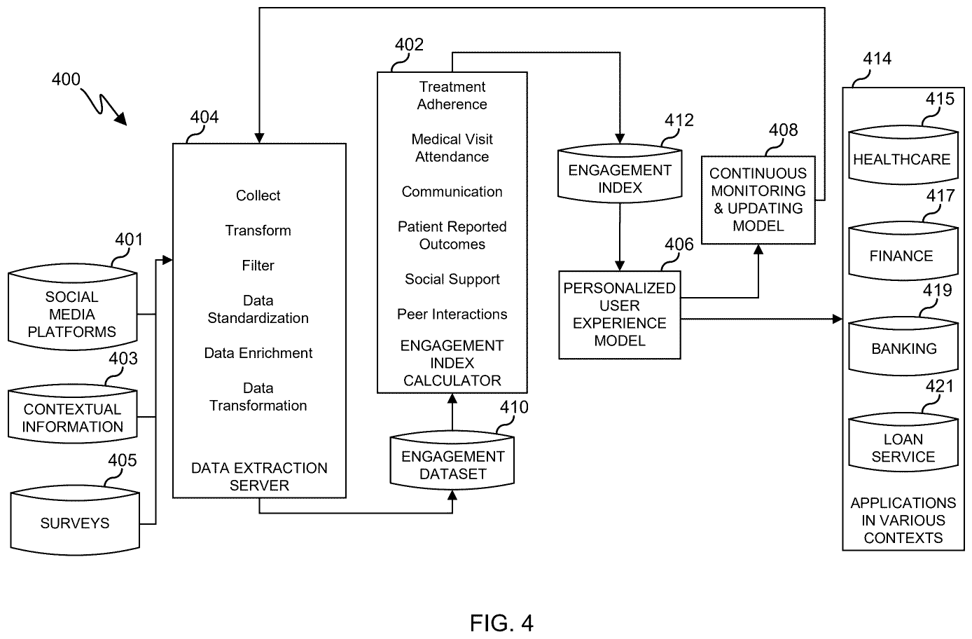

FIG. 4 illustrates an example engagement index system architecture 400 in accordance with this disclosure. For ease of explanation, the architecture 400 shown in FIG. 4 is described as being implemented on or supported by the electronic device 101 in the network configuration 100 of FIG. 1. However, the architecture 400 shown in FIG. 4 could be used with any other suitable device(s) and in any other suitable system(s), such as when the architecture 400 is implemented on or supported by the server 106.

The engagement index system architecture 400 is configured to identify engagement of a consumer in a particular context (e.g., healthcare provider, payer, etc.) over time by integrating the consumer's behavior in different online mediums and physical interactions (such as captured through surveys) with use of algorithms that measure affective, behavioral, and/or cognitive attributes (that may be limited to knowledge of the engagement activity) and captured with attributions such as intensity, frequency, recency, and duration.

For example, as shown in FIG. 4, the architecture 400 includes an engagement index calculator 402, a data extraction server 404, a personalized user experience model 406, and a continuous monitoring and updating model 408. The data extraction server 404 can be connected to various data sources including social media platform sources 401, contextual information sources 403, and survey data sources 405. The data sources may have their data stored in the form of databases. The architecture 400 integrates data from the multiple sources to create a comprehensive engagement index for each consumer. Data can be gathered on user interactions, behaviors, preferences, sentiments, and other relevant information.

The data extraction server 404 can be a dedicated server responsible for extracting relevant data from various sources. The data extraction server 404, using the various data sources, collects, transforms and/or filters the data, and/or performs data standardization, data enrichment, and/or data transformation on the data. For example, the data extraction server 404 performs data acquisition and normalization by collecting data from the multiple sources and performs data preprocessing techniques such as data cleaning, transformation, and normalization to ensure a consistent format and representation across different sources. Using the data extraction server 404, the architecture 400 gathers data from the multiple sources and generates an engagement dataset 410 for each subject individual.

In various embodiments, data collection performed by the data extraction server 404 involves the data extraction server 404 connecting to various data sources, such as databases, APIs, IoT devices, and third-party services, to collect the data. In various embodiments, data transformation performed by the data extraction server 404 involves the data extraction server 404 transforming the collected data into a unified format compatible with other components of the architecture 400, such as the engagement index calculator 402. This can include parsing, decoding, and converting data to ensure seamless integration with the rest of the system.

In various embodiments, data filtering performed by the data extraction server 404 involves the data extraction server 404 filtering the collected data based on predefined criteria or rules, ensuring that only relevant and high-quality data is passed on for further analysis to other components of the architecture 400, such as the engagement index calculator 402. In various embodiments, data enrichment performed by the data extraction server 404 involves the data extraction server 404 enriching the collected data with additional information from external sources, if required, to enhance the context and accuracy of the data fed into the other components of the architecture 400. In various embodiments, data transmission performed by the data extraction server 404 involves the data extraction server 404 securely transmitting the preprocessed data to the other components of the architecture 400.

The pre-processing of the data by the data extraction server 404 thus allows for, before determining the engagement index, the raw data to be collected from the multiple sources and cleaned, transformed, and structured into a format suitable for analysis. The data extraction server 404 may also address missing or incomplete data, such as removing outliers and/or normalizing values to ensure consistent representation across data sources.

The architecture 400 utilizes advanced algorithms to analyze the collected data in the engagement dataset 410 to determine an engagement index 412. For instance, the collected data from the multiple sources is processed and integrated into the unified dataset 410, which is then used as input for the engagement index calculator 402. Determining the engagement index 412 by the engagement index calculator 402 involves the use of advanced algorithms and machine learning techniques to analyze the integrated dataset. These algorithms measure affective, behavioral, and cognitive attributes related to the consumer's engagement in a specific context.

Factors considered in the calculation of the engagement index 412 include the intensity, frequency, recency, and duration of the consumer's interactions. In various embodiments, the engagement index 412 is calculated as a numerical value representing the overall engagement level of the consumer in the given context. The engagement index 412 can be used to identify trends, patterns, and opportunities for intervention, enabling proactive measures to enhance user engagement.

For example, the personalized user experience model 406, which can incorporate various machine learning models, can use the engagement index 412 as a basis for providing a personalized user experience in the respective interaction environment. The architecture 400, using the personalized user experience model 406, can tailor content, offers, and communication strategies based on the consumer's engagement index to improve overall satisfaction and encourage further engagement. The personalized user experience can be applied in various contexts 414, such as healthcare contexts 415, finance contexts 417, banking contexts 419, and loan service contexts 421.

The continuous monitoring and updating model 408 of the architecture 400 also provides for the engagement index to be continuously monitored and updated as new data becomes available, ensuring that it remains adaptive and responsive to changing user behaviors and preferences. For instance, the architecture 400, using the continuous monitoring and updating model 408, can track changes in engagement levels over time, allowing for the identification of emerging trends and potential issues that may require attention. The continuous monitoring and updating model 408 can also create a feedback loop that enables adaptive and dynamic engagement strategies.

Although FIG. 4 illustrates one example of an engagement index system architecture 400, various changes may be made to FIG. 4. For example, various components and functions in FIG. 4 may be combined, further subdivided, replicated, or rearranged according to particular needs. Also, one or more additional components and functions may be included if needed or desired. Further, various components shown in FIG. 4, such as the engagement index calculator 402, the personalized user experience model 406, and/or the continuous monitoring and updating model 408, can be executed by specific electronic devices configured to perform functions related to those components of the architecture 400, or can represent software operations executed by one or more electronic devices.

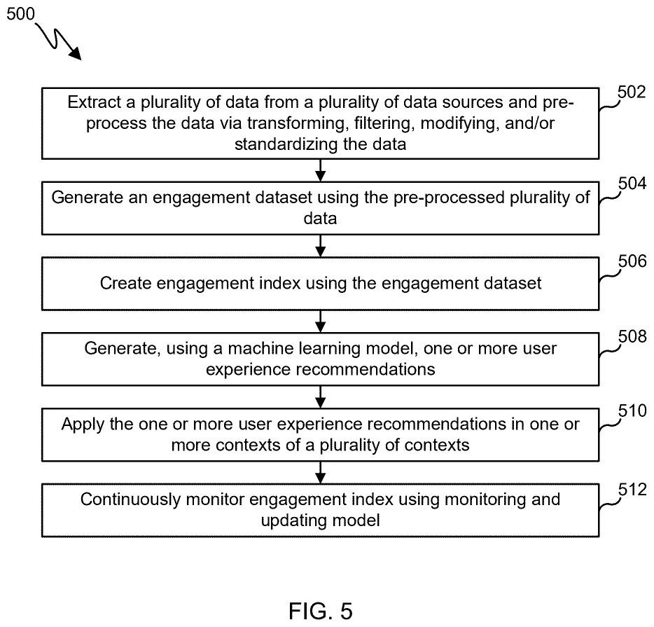

FIG. 5 illustrates an example engagement index method 500 in accordance with this disclosure. For ease of explanation, the method 500 shown in FIG. 5 is described as being performed using the electronic device 101 in the network configuration 100 of FIG. 1. However, the method 500 could be performed using any other suitable device(s), such as the server 106, and in any other suitable system(s).

The method 500 involves identifying engagement of a consumer in a particular context (e.g., healthcare provider, payer, etc.) over time by integrating the consumer's behavior in different online mediums and physical interactions (such as captured through surveys) with use of algorithms that measure affective, behavioral, and/or cognitive attributes (that may be limited to knowledge of the engagement activity) and captured with attributions such as intensity, frequency, recency, and duration.

For example, at step 502, at least one processing device extracts a plurality of data from a plurality of data sources and pre-process the data via transforming, filtering, modifying, and/or standardizing the data, as described with respect to FIG. 4. For instance, a data extraction server, such as the data extraction server 404, can be connected to the various data sources, such as the social media platform sources 401, the contextual information sources 403, and the survey data sources 405. The data sources may have their data stored in the form of databases, and the data extraction server can perform collecting and pre-processing of the data. As described below, the method 500 integrates data from the multiple sources to create a comprehensive engagement index for each consumer. Data can be gathered on user interactions, behaviors, preferences, sentiments, and other relevant information. For example, the pre-processing of the data at step 502 can include transforming and/or filtering the data, and/or performing data standardization, data enrichment, and/or data transformation on the data. This can also include performing data acquisition and normalization by collecting data from the multiple sources and performs data preprocessing techniques such as data cleaning, transformation, and normalization to ensure a consistent format and representation across different sources.

In various embodiments, data collection performed at step 502 involves the data extraction server connecting to various data sources, such as databases, APIs, IoT devices, and third-party services, to collect the data. In various embodiments, data transformation performed by the data extraction server involves the data extraction server transforming the collected data into a unified format compatible with other components used by the method 500, such as an engagement index calculator like the engagement index calculator 402. This can include parsing, decoding, and converting data to ensure seamless integration with the rest of the system.

In various embodiments, data filtering performed by the data extraction server involves the data extraction server filtering the collected data based on predefined criteria or rules, ensuring that only relevant and high-quality data is passed on for further analysis to other components used by the method 500, such as the engagement index calculator. In various embodiments, data enrichment performed by the data extraction server involves the data extraction server enriching the collected data with additional information from external sources, if required, to enhance the context and accuracy of the data fed into the other components used by the method 500. In various embodiments, data transmission performed by the data extraction server involves the data extraction server securely transmitting the preprocessed data to the other components used by the method 500. The pre-processing of the data by the data at step 502 thus allows for, before determining the engagement index, the raw data to be collected from the multiple sources and cleaned, transformed, and structured into a format suitable for analysis. This may also involve addressing missing or incomplete data, such as removing outliers and/or normalizing values to ensure consistent representation across data sources.

At step 504, the at least one processing device generates, such as via the data extraction server, an engagement dataset, such as the engagement dataset 410, using the pre-processed plurality of data. At step 506, an engagement index is created using the engagement dataset. This can include utilizing advanced algorithms to analyze the collected data in the engagement dataset to determine the engagement index. For instance, the collected data from the multiple sources is processed and integrated into the unified engagement dataset, which is then used as input for the engagement index calculator. Determining the engagement index by the engagement index calculator involves the use of advanced algorithms and machine learning techniques to analyze the integrated dataset. These algorithms measure affective, behavioral, and cognitive attributes related to the consumer's engagement in a specific context.

Factors considered in the calculation of the engagement index at step 506 include the intensity, frequency, recency, and duration of the consumer's interactions. In various embodiments, the engagement index is calculated as a numerical value representing the overall engagement level of the consumer in the given context. At step 508, a machine learning model is used to generate one or more user experience recommendations. For example, the machine learning model, which can be the personalized user experience model 406, can use the engagement index to identify trends, patterns, and opportunities for intervention, enabling proactive measures to enhance user engagement. For example, the personalized user experience model, which can incorporate various machine learning models, can use the engagement index as a basis for providing a personalized user experience in the respective interaction environment. The method 500, using the personalized user experience model, can tailor content, offers, and communication strategies based on the consumer's engagement index to improve overall satisfaction and encourage further engagement. At step 510, the personalized user experience recommendations are applied in various contexts, such as healthcare contexts 415, finance contexts 417, banking contexts 419, and loan service contexts 421.

At step 512, the engagement index is continuously monitored and updated, such as via use of a monitoring and updating model like the continuous monitoring and updating model 408 of the architecture 400. This provides for the engagement index to be continuously monitored and updated as new data becomes available, ensuring that it remains adaptive and responsive to changing user behaviors and preferences. For instance, the method 500, using the continuous monitoring and updating model, can track changes in engagement levels over time, allowing for the identification of emerging trends and potential issues that may require attention. The method 500 can also include using the continuous monitoring and updating model to create a feedback loop that enables adaptive and dynamic engagement strategies.

Although FIG. 5 illustrates one example of an engagement index method 500, various changes may be made to FIG. 5. For example, while shown as a series of steps, various steps in FIG. 5 could overlap, occur in parallel, occur in a different order, or occur any number of times (including zero times).

It will be understood that the architectures 200 and 400 shown and described with respect to FIGS. 2 and 4 can be combined as part of one architecture in which the processes and methods facilitated by the architectures 200 and 400 can be performed by the one architecture. For example, in various embodiments, the engagement index calculator 402 of FIG. 4 can be the engagement index calculator 206 of FIG. 2, the data extraction server 404 of FIG. 4 can be the data extraction server 204 of FIG. 2, the engagement dataset 410 of FIG. 4 can be the dataset 214 of FIG. 2, and the continuous monitoring and updating model 408 of FIG. 4 can be, or can perform, the monitoring operation 220 of FIG. 2. That is, the functionalities of these components could be combined and performed by one device or by a one or more devices in cooperation.

Although this disclosure has been described with example embodiments, various changes and modifications may be suggested to one skilled in the art. It is intended that this disclosure encompass such changes and modifications as fall within the scope of the appended claims.

What is claimed is:

1. An engagement operating system comprising:
at least one processing device configured to:
extract a plurality of data from a plurality of data sources and pre-process the plurality of data via transforming, filtering, modifying, and/or standardizing the plurality of data;
determine an engagement index;
identify, using one or more artificial intelligence models, patterns and trends using the engagement index;
generate, using the identified patterns and trends, at least one of:
one or more engagement pathways; or
one or more user experience recommendations; and
continuously monitor the engagement index using a monitoring and updating model to adjust at least one of the one or more engagement pathways or the one or more user experience recommendations.

2. The engagement operating system of claim 1, wherein the at least one processing device is further configured to:
generate an N-gram dataset using the pre-processed plurality of data; and
store the N-gram dataset in an N-gram repository.

3. The engagement operating system of claim 2, wherein, to generate the N-gram dataset, the at least one processing device is further configured to:
slide a fixed-size window over the pre-processed plurality of data to capture data combinations within the fixed-size window; and
create sequences of features representing a fixed-length combination of consecutive elements in the pre-processed plurality of data.

4. The engagement operating system of claim 2, wherein the at least one processing device is further configured to:
determine the engagement index using N-grams in the N-gram repository; and
identify, using the one or more artificial intelligence models, patterns and trends in the N-gram dataset.

5. The engagement operating system of claim 4, wherein the engagement index representing a numerical value reflecting a composite effect of various factors on fitness and engagement related to a user.

6. The engagement operating system of claim 4, wherein the at least one processing device is further configured to transmit data on the one or more engagement pathways to an electronic device.

7. The engagement operating system of claim 2, wherein the at least one processing device is further configured to store on a blockchain network at least a portion of one or more of:
the pre-processed plurality of data;
the N-gram dataset;
the engagement index;
the one or more engagement pathways; or
the one or more user experience recommendations.

8. The engagement operating system of claim 1, wherein the at least one processing device is further configured to:
generate an engagement dataset using the pre-processed plurality of data; and
create the engagement index using the engagement dataset.

9. The engagement operating system of claim 1, wherein the at least one processing device is further configured to apply the one or more user experience recommendations in one or more contexts of a plurality of contexts.

10. The engagement operating system of claim 1, wherein the at least one processing device is further configured to generate, using the identified patterns and trends, both the one or more engagement pathways and the one or more user experience recommendations.

11. A method of an engagement operating system, the method comprising:
extracting a plurality of data from a plurality of data sources and pre-processing the plurality of data via transforming, filtering, modifying, and/or standardizing the plurality of data;
determining an engagement index;
identifying, using one or more artificial intelligence models, patterns and trends using the engagement index;
generating, using the identified patterns and trends, at least one of:
one or more engagement pathways; or
one or more user experience recommendations; and
continuously monitoring the engagement index using a monitoring and updating model to adjust at least one of the one or more engagement pathways or the one or more user experience recommendations.

12. The method of claim 11, further comprising:
generating an N-gram dataset using the pre-processed plurality of data; and
storing the N-gram dataset in an N-gram repository.

13. The method of claim 12, wherein generating the N-gram dataset comprises:
sliding a fixed-size window over the pre-processed plurality of data to capture data combinations within the fixed-size window; and
creating sequences of features representing a fixed-length combination of consecutive elements in the pre-processed plurality of data.

14. The method of claim 12, further comprising:
determining the engagement index using N-grams in the N-gram repository; and
identifying, using the one or more artificial intelligence models, patterns and trends in the N-gram dataset.

15. The method of claim 14, wherein the engagement index representing a numerical value reflecting a composite effect of various factors on fitness and engagement related to a user.

16. The method of claim 14, further comprising transmitting data on the one or more engagement pathways to an electronic device.

17. The method of claim 12, further comprising storing on a blockchain network at least a portion of one or more of:
the pre-processed plurality of data;
the N-gram dataset;
the engagement index;
the one or more engagement pathways; or
the one or more user experience recommendations.

18. The method of claim 11, further comprising:
generating an engagement dataset using the pre-processed plurality of data; and
creating the engagement index using the engagement dataset.

19. The method of claim 11, further comprising applying the one or more user experience recommendations in one or more contexts of a plurality of contexts.

20. The method of claim 11, further comprising generating, using the identified patterns and trends, both the one or more engagement pathways and the one or more user experience recommendations.
